(19) United States
(12) Patent Application Publication
(10) Pub. No.: US 2025/0154551 A1
(43) Pub. Date: May 15, 2025

(54) METHOD AND SYSTEM FOR DETECTING AND IDENTIFYING A MICRO-ORGANISM CONTAINED IN A SAMPLE

(71) Applicant: COMMISSARIAT A L'ENERGIE ATOMIQUE ET AUX ENERGIES ALTERNATIVES, Paris (FR)

(72) Inventors: Thibaut BABIN, Grenoble Cedex 9 (FR); Maxime GOUGIS, Grenoble Cedex 9 (FR); Pascal MAILLEY, Grenoble Cedex 9 (FR); Pierre MARCOUX, Grenoble Cedex 9 (FR)

(73) Assignee: COMMISSARIAT A L'ENERGIE ATOMIQUE ET AUX ENERGIES ALTERNATIVES, Paris (FR)

(21) Appl. No.: 18/724,711

(22) PCT Filed: Dec. 29, 2022
(86) PCT No.: PCT/FR2022/052517

§ 371 Date: Jan. 10, 2025

(30) Foreign Application Priority Data
Dec. 30, 2021 (FR) 2114688

(51) Int. Cl. C12Q 1/04; C12M 1/34; G01N 27/30; G01N 27/327; G01N 27/416

(52) U.S. Cl. CPC C12Q 1/04; C12M 41/46; G01N 27/301; G01N 27/327; G01N 27/416

(57) ABSTRACT

The present invention relates to a method for detecting and identifying a micro-organism contained in a sample comprising the steps consisting in (a) placing the sample, in a liquid culture medium, in the presence of at least one measurement electrode and a reference electrode and under conditions suitable for growth of the micro-organisms, (b) measuring, over time, the potential difference between said at least one measurement electrode and the reference electrode, (c) optionally computing the derivative of the potential difference based on the measurement performed in step (b) by way of which the profile of the derivative of the potential is obtained, and (d) detecting and identifying a micro-organism contained in said sample either based on the profile of the potential obtained in step b), or based on the profile obtained in step c). The present invention also relates to a system that capable of being implemented in this method.

### TECHNICAL FIELD

The present invention belongs to the technical field of diagnostic microbiology and more particularly to the field of detection and identification of micro-organisms present in a liquid or solid sample.

The main advance of the present invention lies in the use of electrochemistry for the identification, with no a priori, of a micro-organism such as a pathogen. The applications of the present invention relate in particular to sterility tests in the health environment but also in other environments like the pharmaceutical, biomedical, environmental or agri-food environments.

### PRIOR ART

In clinical microbiology, and more particularly in the field of blood culture, the operator samples about 10 mL of blood on a patient. This test sample is placed in a flask containing between 30 and 40 mL of a culture medium conducive to bacterial growth. The principle of detection is based on bacterial breathing, by multiplying the bacteria will produce an amount of detectable carbon dioxide.

For example, the bottom of the flask may be composed of a polymer, which will change color or fluorescence according to the CO₂ concentration of the liquid. The blood culture controllers then allow stirring the flasks at 37° C. while using an optical method in order to verify the variation in color or fluorescence of the polymer.

Once the flask has been determined as positive, the operator should sample a small amount of the culture broth in order to perform Gram staining. This manipulation is not automated and requires dangerous products such as, for example, crystal violet or gentian violet, Lugol solution (iodine), alcohol, acetone or fuchsin. After this first orientation, the identification is refined with the use of a MALDI-TOF (“Matrix Assisted Laser Desorption/Ionization-Time Of Flight”) mass spectrophotometer and then an antibiogram.

It arises from this method with several long steps that the time between the assay and the result lasts, at best, several tens hours. This considerable delay cannot be associated with an absence of treatment, a wide-spectrum antibiotherapy is started without waiting for the result of the blood culture. Yet, antibiotics that are barely suitable for the pathogen, if not at all, could lead to an antibioresistance, one of the greatest dangers for world health.

Nowadays, blood culture controllers or CMBCS for “Continuous Monitoring Blood Culture System”, are all systems which merely deal with the sterility test alone: they detect the presence of a micro-organism, even if it is one single cell, without giving any information as to its nature like eukaryotic or prokaryotic, Gram+ or Gram−, or its species.

Mention may be made of radiometric respirometry systems (Bactec™ 460, BD), infrared CO₂ detection systems (Bactec™ 660/730/860, BD), fluorescence CO₂ detection systems (Bactec™ 9240, BD), for colorimetric CO₂ detection systems (BacT/ALERT®, bioMerieux) or manometric detection systems (VersaTREK™, ThermoScientific and SIGNAL™, ThermoScientific).

Few automated blood culture systems have proposed both the detection and the identification, or at least, an orientation. Mention may be made of:
in the gas phase: the use of a CSA (“Colorimetric Sensor Array”), an artificial nose with colorimetric transduction, inserted into the stopper of the flask [1];
in the liquid phase: the use of antibody networks on an SPR prism in contact with the blood culture [2].

In electrochemistry, it is common to use pH-sensitive electrodes. Several materials are known for their characteristic potential variation in a given pH range, including conductive polymers like polyaniline and metal oxides like iridium oxide.

The International application WO 2021/032654 A1 [3] provides a device of the autonomous controller type allowing detecting the presence of micro-organisms in a sample by monitoring the variations in pH by electrochemistry.

Most research teams working on these technologies use electrochemistry for the a priori detection of a pathogen. Indeed, thanks to the use of a probe immobilized on an electrode and by selecting the suitable measurement method, it is possible to detect the coupling between the probe and the micro-organism.

The probe depends on the pathogen, the medium and the used technique. It is possible to graft an antibody, a bacteriophage, a thiol probe, an aptamer, a DNA probe, an RNA probe or a protein-specific probe in order to detect a biological mechanism specific to the bacterium of interest.

The used main techniques are amperometry consisting in detecting a variation in current and impedance spectroscopy consisting in detecting a modification of the resistance of the electrode.

The major obstacle to the use of these technologies in the field of diagnostic microbiology for the detection step is their a priori detection, which cannot be considered in clinical terms. In addition, although these types of electrodes allow discriminating pathogens between that one looked for and other “parasitic” microbes, it is not possible to identify a pathogen other than that one looked for.

The Inventors have set themselves the aim of providing a method that addresses the technical problems of the methods of the prior art, i.e. a method that is easy to implement, fast, effective, allowing detecting and then identifying a micro-organism contained in a liquid or solid sample.

### DISCLOSURE OF THE INVENTION

The present invention allows achieving the aim that the Inventors have set. Indeed, these have demonstrated that it was possible to provide a method that is easy to implement, fast, effective, thanks to which it is possible to detect and then identify a micro-organism contained in a liquid or solid sample, using electrochemistry.

The invention is based on an electrochemical signal variation, in particular on the evolution of the electrochemical potential and, in particular, on the evolution of the derivative of the electrochemical potential with respect to time of at least one and typically of several electrodes of different materials. With a suitable processing of the signal, it is possible to detect the growth of a micro-organism like a bacterial growth and then to identify the micro-organism at different degrees of accuracy. Hence, this allows reducing the time before the result, reducing the working time and the risks for the operator.

The method object of the invention may be implemented on the instrumented flask as described in the International application WO 2021/032654 A1 [3] in which electrodes composed of different materials enabling a faster detection are placed for two reasons:
1) the flask is already instrumented and does not require a bulky controller, the transport time usually required is no longer necessary, and
2) the measurement is performed directly in the flask and not via a carbon dioxide-sensitive polymer.

Thus, the present invention relates to a method for detecting and identifying a micro-organism contained in a sample comprising the steps of
a) placing the sample, in a liquid culture medium, in the presence of at least one measurement electrode and a reference electrode and under conditions suitable for the growth of the micro-organisms,
b) measuring, over time, the potential difference between the measurement electrode(s) and the reference electrode,
c) optionally calculating the derivative of the potential difference from the measurement performed in step b) whereby the profile of the derivative of the potential is obtained, and
d) detecting and identifying a micro-organism contained in said sample either from the profile of the potential obtained in step b) or from the profile obtained in step c).

In a first embodiment, the present invention relates to a method for detecting and identifying a micro-organism contained in a sample comprising the steps of
a) placing the sample, in a liquid culture medium, in the presence of at least one measurement electrode and a reference electrode and under conditions suitable for the growth of the micro-organisms,
b) measuring, over time, the potential difference between the measurement electrode(s) and the reference electrode, and
d) detecting and identifying a micro-organism contained in said sample from the profile of the potential obtained in step b).

In this embodiment, the detection and identification of the micro-organism contained in the sample is realized from the potentiometric signal obtained in step b).

In a second embodiment, the present invention relates to a method for detecting and identifying a micro-organism contained in a sample comprising the steps of
a) placing the sample, in a liquid culture medium, in the presence of at least one measurement electrode and a reference electrode and under conditions suitable for the growth of the micro-organisms,
b) measuring, over time, the potential difference between the measurement electrode(s) and the reference electrode,
c) calculating the derivative of the potential difference from the measurement performed in step b) whereby the profile of the derivative of the potential is obtained, and
d) detecting and identifying a micro-organism contained in said sample from the profile obtained in step c).

The sample implemented in the context of the method of the invention may be any sample usually sterile but which could contain micro-organisms following a particular event like a contamination, an infection, a pathology or a dysfunction of a filtration, decontamination or cleaning system. This sample may be in a liquid or solid form.

The sample implemented in the method according to the invention is advantageously selected from the group consisting of a biological fluid; water like, for example, sterile grade filtered water, in particular used in medical facilities and production, underground water, source water or surface water; a sample from a liquid industrial effluent; a sample originating from an air filtration; a sample in a food, cosmetic or pharmaceutical product and a sample on an object.

In a particular embodiment, the biological fluid is advantageously selected from the group consisting of blood such as whole blood or anti-coagulated whole blood, blood serum, blood plasma, lymph, tears, sperm, urine, milk, cerebrospinal fluid, interstitial fluid, an articular liquid, a pericardial fluid, a fluid isolated from bone marrow, a cell extract, a tissue extract, an extract of organs and one of mixtures thereof. Thus, the biological fluid may be any fluid naturally secreted or excreted from a human or animal body or any fluid recovered, from a human or animal body, by any technique known to a person skilled in the art such as extraction, sampling, puncture or washing. The steps of recovering and isolating these different fluids from the human or animal body are carried out prior to the implementation of the method according to the invention.

In a particular embodiment, the liquid industrial effluent in which the sample is performed may be an effluent from an agri-food, pharmaceutical or cosmetic industry.

In a particular embodiment, the object on which sampling is performed may be selected from among large-sized facilities like an industrial object like an electronic apparatus or a machine used in the agri-food, pharmaceutical or cosmetic industries, a tank, a restaurant kitchen, a cold chamber, a sanitary ware, a container, and small-sized objects like medical devices or hoses.

In the context of the present invention, by “sampling”, it should be understood any type of sample collection, for example, by contact, scraping, swabbing, drilling, cutting, punching, grinding, washing, rinsing, suction, puncture or pumping.

In a more particular embodiment, the sample implemented in the method according to the invention is blood like human or animal blood. The latter is normally sterile but may contain micro-organisms like bacteria during non-pathological or pathological bacteremia. Indeed, bacteria may be present in the blood following theoretically inoffensive procedures such as toothbrushing, dental care or medical procedures involving probes or catheters. We then talk about non-pathological bacteremia. In turn, the pathological bacteremia corresponds to the presence of bacteria in blood following a pneumonia, a wound or a urinary infection.

Among the micro-organisms contained or likely to be contained in the sample, prokaryotic micro-organisms such as archaea or Gram+ or Gram− bacteria are found as well as eukaryotic micro-organisms such as yeasts and other microscopic fungi and protists like algae or protozoa.

As illustrative and non-limiting examples of micro-organisms contained or likely to be contained in the sample implemented in the context of the invention, mention may be made of bacteria of the Enterobacteriaceae family, bacteria of the Pseudomonadaceae family, bacteria of the genus Staphylococcus, bacteria of the genus Streptococcus, bacteria of the genus Campylobacter, bacteria of the genus Haemophilus, bacteria of the genus Anaerococcus, bacteria of the genus Bacteroides, bacteria of the genus Acinetobacter, bacteria of the genus Stenotrophomonas, bacteria of the genus Achromobacter, bacteria of the genus Fusobacterium, bacteria of the genus Pasteurella, bacteria of the genus Bacillus, bacteria of the genus Listeria, bacteria of the genus Clostridium, bacteria of the genus Mycobacteria, bacteria of the genus Enterococcus and bacteria of the genus Pasteurella, yeasts of the genus Candida, yeasts of the genus Saccharomyces, fungi of the genus Aspergillus, fungi of the genus Penicillium and/or archaea of the genus Methanobrevibacter.

In addition, among the bacteria of the Enterobacteriaceae family contained or likely to be contained in the sample implemented in the context of the invention, mention may be made of bacteria of the genera Escherichia, Salmonella, Shigella, Enterobacter, Klebsiella, Serratia, Proteus, Morganella, Yersinia, Citrobacter and Providencia.

Prior to contacting with the liquid culture medium in the presence of the electrodes, i.e. prior to step a) of the method according to the invention, the sample may be subjected to a preparation step by means of any process for preparing a sample likely to contain micro-organisms, known to a person skilled in the art. In particular, this step may consist, in a non-exhaustive manner, of a dilution, a concentration, a division into portions which may be equivalent, or not, or a suspension.

Typically, during step a) of the method according to the invention, the sample is diluted in the liquid culture medium, when this sample is liquid. Alternatively, when it is solid, it is suspended in the liquid culture medium.

The liquid culture medium implemented during step a) of the method according to the invention may be any liquid medium suitable for the culture of micro-organisms, known to a person skilled in the art. By “suitable for culturing micro-organisms”, it should be understood a preparation within which the micro-organisms can multiply. This medium comprises a carbon source, like glucose, acetate or glycerol; a source of nitrogen, like ammonium, nitrates or amino acids; and salts and/or trace elements and vitamins for the growth of micro-organisms. In particular, this liquid culture medium may be not selective for a particular type of micro-organisms. Alternatively, it may be selective for a particular type of micro-organisms.

As examples of liquid culture media that can be used during step a) of the method according to the invention, mention may be made of the NB1 (“Nutrient Broth No. 1”) culture medium, the NB2 (“Nutrient Broth No. 2”) culture medium, the Mueller Hinton culture medium, the LB (“Lysogen Broth”, also known as “Luria Bertani”) culture medium, the TSB (“Tryptic Soybean Broth”) culture medium, the BHI (“Brain Heart Infusion”) culture medium and the BACT/ALERT® culture medium. In particular, these culture media are available from the companies ThermoFisher, Bio-Rad Laboratories, bioMerieux and Sigma Aldrich.

During step a) of the method according to the invention, conditions suitable for the growth of the micro-organisms are implemented. In other words, during step a) of the process according to the present invention, the micro-organisms are kept in an environment which enables them to increase in number by cell division. This environment comprises not only the culture medium in which the sample has been diluted or resuspended, but also other conditions such as a temperature and an atmosphere that enable cell growth.

Typically, the temperature implemented during step a) is between 20° C. and 40° C. Particular examples of temperature implemented in step a) are 20° C., 25° C., 30° C., 32° C., 37° C. and 39° C.

As regards the atmospheric conditions, step a) may be performed under an oxygen-free atmosphere, under air, in air +5% of CO₂ or under a gas mixture composed of 5% of O₂, 5% of CO₂ and 90% of N₂. An oxygen-free atmosphere, i.e. an anoxic atmosphere, should be preferred when the micro-organism(s) contained or likely to be contained in the sample grow under strict anaerobic conditions. Advantageously, as explained before, the method according to the invention is carried out in an instrumented flask as described in the International application WO 2021/032654 A1 [3], the atmospheric conditions therefore correspond to the air contained in the flask following hermetic closure thereof by a stopper.

During step a) of the method according to the invention, the culture medium comprising the sample likely to contain or containing micro-organisms is in contact with at least two electrodes. These electrodes correspond to at least one measurement electrode, also referred to by the expression “working electrode” and a reference electrode.

In a particular embodiment, at least two, at least three, at least four, at least five, at least six, at least seven, at least eight, at least nine, at least ten measurement electrodes, identical or different, may be implemented. The experimental part hereinafter presents a more particular embodiment with four different pairs of measurement electrodes and a reference electrode.

By “different measurement electrodes”, it should be understood electrodes whose chemical nature differs. Any measurement electrode known to a person skilled in the art can be used in the context of the present invention.

Thus, the measurement electrode(s) used in the method according to the invention may be selected from the group consisting of a solid electrode made of metal, a glass electrode, an electrode made of glassy carbon, an electrode made in the form of a deposit of a conductive ink and an electrode made in the form of an electrodeposit of a conductive element.

Any conductive ink known to a person skilled in the art may be implemented in the context of the invention. In a particular embodiment, the used conductive ink is an ink based on metal ions or on conductive organic polymers such as polythiophene (PT), polyaniline (PANI) optionally doped with dodecylbenzenesulfonic acid (DBSA), poly (3,4-ethylenedioxythiophene) coupled to sodium polystyrene sulfonate (PEDOT:PSS), polypyrrole or polyphthalocyanine. In another particular embodiment, the conductive ink used in the context of the invention is a carbon ink optionally comprising an additional conductive element.

By “conductive element”, it should be understood an element selected from the group consisting of conductive organic polymers such as those listed hereinabove, and metal-based compounds like, for example, a metal oxide such as iridium oxide (IrOx), a metal-based pigment such as Prussian blue (Fe(III) ferrocyanide) or an organic or organo-inorganic catalyst such as cobalt phthalocyanine.

Any reference electrode conventionally used in electrochemistry may be implemented in the context of the invention. Advantageously, the reference electrode used in the context of the invention is made in the form of a deposit of an Ag/AgCl ink covered with a polymer layer such as a polyurethane layer, to avoid the diffusion of silver particles and an inhibition of the growth of the micro-organisms.

It is also possible to place a counter-electrode in contact with the liquid culture medium during the method according to the invention. The presence of a counter-electrode is in no way mandatory.

Everything described in the international application WO 2021/032654 A1 [3] with regards to the measurement and reference electrodes also applies to the present invention like, for example, the two-part organization of the electrodes and the implemented electrical contacts.

Advantageously, the liquid culture medium and the electrodes are sterile or have been sterilized before any contact with the sample, i.e. prior to step a) of the method. The culture medium may be sterilized via different techniques known in the state of the art such as, yet without limitation, the use of aseptic techniques to autoclave and/or prepare the culture medium. In particular, the autoclaving technique can be selected to sterilize the electrodes optionally in the presence of the liquid culture medium intended to receive the sample.

Step b) of the method according to the invention corresponds to monitoring the evolution, in the liquid culture medium, of the potential difference between the measurement electrode(s) and the reference electrode.

In a particular embodiment, this measurement of the potential difference is done continuously. Indeed, it is possible using a multichannel potentiostat, to measure the potential difference between the measurement electrode(s) and the reference electrode continuously (modulo the sampling period of the potentiostat—for example a few ms).

In another particular embodiment, this measurement of the potential difference is done discontinuously.

To do so, the potential difference is measured at a plurality of distinct time points. In other words, step b) consists in measuring the potential difference between the measurement electrode(s) and the reference electrode at a plurality of time points whereby potential measurement points are obtained.

By “plurality of distinct time points”, it should be understood more than 2, in particular more than 5, in particular more than 10 and, more particularly, more than 20 distinct time points. These different time points may be selected at regular intervals or at irregular intervals. In one particular implementation, the measurements are performed at regular intervals. A person skilled in the art should know how to determine, without involving any inventive step, the most suitable time interval according to the sample to be studied and the micro-organisms likely to be present in the latter. As a particular example, this interval may be comprised between 1 min and 30 min, in particular between 2 min and 15 min and, in particular, in the range of 5 min (5 min±1 min). In another particular implementation, the measurements are performed at irregular intervals. Indeed, it is possible to consider performing a measurement according to the time drift of the signal, thereby allowing measuring more points in the areas of high variation versus the periods of stagnation of the potential.

In a particular embodiment, and whether the measurement is continuous or discontinuous, it is possible to carry out the first measurement of the potential difference at the time point t₁ just after step a) of the method of the invention i.e. 1 min, 2 min, 3 min, 4 min or 5 min after setting the sample into contact in the liquid culture medium in the presence of the electrodes.

In another particular embodiment, and whether the measurement is continuous or discontinuous, it is possible to carry out the first potential measurement at the time point t₁ at least 1 h after setting the sample into contact in the liquid culture medium in the presence of the electrodes.

The interval between the time point t₁ of the first measurement and the time point t_{d} of the last potential measurement during step b) is variable and depends in particular on the sample to be studied, the micro-organisms likely to be present in the latter and the time point t₁. Advantageously, the time point t_{d} of the last potential measurement is carried out at least 14 h, at least 16 h, at least 18 h, at least 20 h or at least 22 h after step a).

Advantageously, the operating conditions during step b) in terms of temperature and atmospheric conditions are identical to those implemented during step a) of the method according to the invention as described before.

Moreover, the culture medium may be subjected to stirring during steps a) and/or b) of the method according to the invention. This stirring during step a) enables a homogeneous mixing of the sample with the culture medium. During step b) and in particular in the presence of micro-organisms, this stirring promotes a uniform composition of the culture medium and prevents any deposition of micro-organisms on the electrodes. Any known technique for stirring a medium not interfering with the measurement of the potential can be used in the context of the present invention. In particular, this stirring is as considered in the International application WO 2021/032654 A1 [3].

In the first embodiment as defined before, the potentiometric signals obtained in step b) are used to detect but above all identify the micro-organism(s) in culture.

Indeed, the different inflection points of the potentiometric signal obtained during the growth of the micro-organisms originating from the sample in the culture medium depend on both the selected material of the measurement electrode and the micro-organism itself. This type of curve allows obtaining information on the nature of the micro-organism(s) in culture. In other words, the potentiometric signal obtained in step b) allows, alone i.e. not combined with any other information, in particular electrochemical information, identifying the micro-organism(s) in culture.

In the second embodiment as defined before, step c) consists in continuously calculating (case of a continuous measurement of the potential difference during step b)) or calculating from the different potential measurements obtained in step b) (case of a discontinuous measurement of the potential difference during step b)), the derivative of the potential.

Indeed, in order to more clearly observe the start of the potential variations when micro-organisms originating from the sample grow in the culture medium, the derivative of the potential difference for each electrode is used. Thus, the rate of modification of the potential is observed, which may be more relevant and readable than the raw potentiometric curves.

In this particular embodiment and in the case where the measurement of the potential difference during step b) is discontinuous, the calculation of the derivative of the potential difference may be done in a sliding manner over a predetermined number of measurement points obtained in step b). In other words, the derivative of the smoothed potential difference is calculated over a predetermined number of potential difference measurement points (sliding average over a predetermined number of potential difference measurement points).

Typically, the pre-determined number of measurement points is equal to 2, 3, 4, 5, 6, 7, 8, 9, 10, 11, 12, 13, 14 or 15. Advantageously, the pre-determined number of measurement points is equal to 5 and that being so, in particular when the interval between two consecutive potential measurement points is in the range of 5 min.

By “in a sliding manner”, it should be understood that the calculation of the derivative is carried out for a pre-determined number n of potential measurement points by calculating the first value of the derivative corresponds to the average of the derivatives of the potential measurement points from p₁ to pₙ, the second value of the derivative corresponds to the average of the derivatives of the potential measurement points from p₂ to pₙ₊₁, the third value of the derivative corresponds to the average of the derivatives of the potential measurement points from p₃ to pₙ₊₂, and so on.

Step c) of the method according to the invention may be carried out once step b) is completed, i.e. for a discontinuous measurement, once all of the potential measurement points are completed. Alternatively, step c) of the method according to the invention may be carried out at the same time as step b) of the method and, for a discontinuous measurement, once the first pre-determined number of potential measurement points obtained. The calculation carried out in step c) i.e. the data processing used in step c) generates a n-dimension response corresponding to the profile of the derivative of the potential difference.

It is also possible to generate a n-dimension response corresponding to the profile of the potential difference (or profile of the potential), in the context of the first embodiment as defined before.

From this n-dimension response, it is possible to extract a normalized profile using methods such as PCA (“PluriComponent Analysis”), MDS (“MultiDimensional Scaling”) and neural networks or Fourier transform to obtain a reduced dimensionality.

In other words, the method according to the invention comprises an additional step consisting in obtaining a normalized profile of the derivative of the potential from the profile of the derivative obtained in step c).

In the context of the first embodiment as defined before, the method according to the invention may also comprise an additional step consisting in obtaining a normalized profile of the potential from the electrochemical profile obtained in step b).

In the context of the first embodiment as defined before, the detection of the presence, or not, of micro-organisms in the sample may be done by comparing the profile of the potential or optionally the normalized profile of the potential with the profile of the potential or optionally the normalized profile of the obtained potential with a negative control i.e. devoid of micro-organisms and under the same operating conditions and in particular the same working electrode(s), the same reference electrode and the same culture medium.

In the context of the second embodiment as previously defined, the detection of the presence, or not, of micro-organisms in the sample may be done by comparing the profile of the derivative of the potential or optionally the normalized profile of the derivative of the potential with the profile of the derivative of the potential or optionally the normalized profile of the derivative of the obtained potential with a negative control, i.e. devoid of micro-organisms and under the same operating conditions and in particular the same working electrode(s), the same reference electrode and the same culture medium.

In the context of the first embodiment as previously defined, the identification of a particular micro-organism may be done by comparing the profile of the potential or optionally the normalized profile of the potential with different profiles of the potential or optionally different normalized profiles of the obtained potential, each, with a known micro-organism and under the same operating conditions and in particular the same working electrode(s), the same reference electrode and the same culture medium.

In the context of the second embodiment as previously defined, the identification of a particular micro-organism may be done by comparing the profile of the derivative of the potential or optionally the normalized profile of the derivative of the potential with different profiles of the derivative of the potential or optionally different normalized profiles of the derivative of the obtained potential, each, with a known micro-organism and under the same operating conditions and in particular the same working electrode(s), the same reference electrode and the same culture medium.

The present invention also relates to a system for detecting and identifying a micro-organism contained in a sample likely to be implemented during the method as defined before comprising
i) a device configured for cell culture in a liquid medium comprising at least one measurement electrode and a reference electrode,
ii) a unit for measuring the potential difference between the measurement electrode(s) and the reference electrode and configured to measure the potential difference, continuously and/or at a plurality of time points,
iii) an electronic unit for acquiring the measurement of the potential difference, connected to the potential measurement unit,
iv) an electric power supply unit for powering the electronic acquisition unit, and
v) a processing unit connected to the electronic acquisition unit and configured to process the measurement of the potential difference and optionally to calculate, from the latter, the derivative of the potential difference and including a module for detecting and identifying a micro-organism by analysis of the potential difference or optionally by analysis of the derivative of the potential difference.

In a particular embodiment, the processing unit is configured to process the measurement points of the potential difference and calculate, from these, the derivative of the potential difference and/or configured to process the continuous measurement of the potential difference and calculate, from the latter, the derivative of the potential difference.

Other features and advantages of the present invention will appear better to a person skilled in the art upon reading the examples hereinbelow given for illustrative and non-limiting purposes, with reference to the appended figures.

### BRIEF DESCRIPTION OF THE DRAWINGS

FIG. 1 shows the potentiometric curves obtained in human blood after inoculation, at t=0, of 38 cfu (“colony-forming units”) of Escherichia coli—ATCC 8739 (PANI=polyaniline/IrOx=iridium oxide/P2=SunChemical Gwent C2070424P2 ink/BQ=Dupont BQ242 ink).

FIG. 2 shows the potentiometric curves obtained in human blood in the absence of any micro-organism (PANI=polyaniline/IrOx=iridium oxide/P2=SunChemical Gwent C2070424P2 ink/BQ=Dupont BQ242 ink).

FIG. 3 shows the smoothed derivatives of the potential of the polyaniline-based electrode, obtained in human blood after growth of different bacteria and for a negative control.

FIG. 4 shows the smoothed derivatives of the potential of the polyaniline-based electrode, obtained in human blood after growth of all of the tested bacteria, the time being normalized to center the maxima.

FIG. 5 shows the 3D space enabling the orientation of the identification of the bacteremia from the obtained potential derivatives.

FIG. 6 shows the 2D space enabling the orientation of the identification of the bacteremia from the obtained potential derivatives.

FIG. 7 shows the module as a function of frequency, calculated from the Fourier transform of the smoothed derivative of the potential of the polyaniline-based electrode, obtained in human blood after growth of all of the tested bacteria.

FIG. 8 shows the rate of correct Gram typing (%) relative to the additional incubation time after positivity of the culture from the obtained potential derivatives.

### DETAILED DISCLOSURE OF PARTICULAR EMBODIMENTS

### I. Manufacture of the Implemented Device.

In order to obtain several electrochemical signals and therefore several electrochemical signatures for each pathogen, several electrode materials have been used.

The measurement device is a printed circuit board PCB (“Printing Circuit Board”) platform, with 10 electrodes made of gold: 8 working electrodes and a reference electrode. The materials, in the form of ink, are deposited over gold and then dried in an oven, at 80° C., for 2 h. The measurement device may optionally comprise a counter-electrode like a counter-electrode made of a commercial carbon ink (Dupont BQ242).

### I.1. Working Electrodes.

Each electrode is made in duplicate on the platform in order to overcome any problems on a deposit. Hence, there are 4 different inks:
i) polyaniline (PANI), doped with dodecylbenzenesulfonic acid (DBSA) (Pani-DBSA ink from Rescoll);
ii) iridium oxide (IrOx), metal oxide, integrated into a carbon ink (Dupont BQ242);
iii) SunChemical Gwent C2070424P2, commercial carbon ink with Prussian blue (Fe (III) ferrocyanide); and
iv) commercial carbon ink (Dupont BQ242).

The PANI and IrOx inks have a known and stable sensitivity to pH variations whereas the SunChemical Gwent C2070424P2 and Dupont BQ242 are selected for the detection of other chemical reactions within the medium.

Each working electrode has a sub-layer of Dupont BQ242 carbon ink in order to improve the mechanical strength of the deposit.

### I.2. Reference Electrode.

On the gold electrode provided to this end, a sub-layer of Dupont BQ242 carbon ink then a layer of Ag/AgCl ink are deposited. The Ag/AgCl ink is commonly used as a material for a pseudo-reference electrode.

This electrode being particularly sensitive to oxidative stress induced by the sterilization method used (the autoclave), it is also protected by a polyurethane membrane which is left to dry for 24 h, at ambient temperature, after deposition.

### I.3. Manufacture of the Flask.

Once the platform has been prepared as explained hereinabove, it is integrated into a polypropylene flask Azlon®. The slot used to make the connector pass outside the flask is sealed by three successive methods:
addition of a LOCTITE® 406 ™ cyanoacrylate glue, after application of an activator,
addition of a fast-setting RS PRO epoxy glue, and
masking of the glue joints with a Kapton® adhesive tape.

Thanks to these precautions, the flask is completely sealed and experiments without any risk of contamination are possible.

### II. Preparation of the Experiment.

### II.1. Prior Sterilization.

Once the flask has been filled with about 30 mL of a BACT/ALERT® culture medium, it is autoclaved in a Tuttnauer 2540 EL autoclave through a “Liquid” cycle. Hence, it undergoes an increase in temperature up to a plateau at 121° C. for 15 min This method allows ensuring the sterility of the electrodes and of the culture medium.

### II.2. Seeding Protocol.

Under a Microbiology Safety Station (MSS), 10 mL of blood are added to the flask. If the selected experiment is a negative control (with no bacteria) then the measurement is launched.

Otherwise, the flask is seeded with a determined amount of bacteria according to the protocol explained hereinbelow, all manipulations taking place under a MSS with the safety steps in force.

Bacterial colonies are added to a flask filled with 3 mL of a pharmaceutical diluent (European Pharmacopeia diluent—tube 9 mL, VWR AX011144). The turbidity of this mixture is measured on a regular basis by a turbidimeter in order to reach a value of 0.5 McFarland.

The addition of the colonies is performed using a microstreaker (or inoculation loop) from a culture on a Petri dish having been prepared and then placed at 37° C. for 24 h.

Once the value of 0.5 McFarland has been reached, a cascade dilution is performed in a pharmaceutical diluent until reaching the desired theoretical bacterial concentration.

A volume of 0.4 mL of this last dilution is sampled and then added to the measurement flask filled beforehand with 30 mL of a BACT/ALERT culture medium and 10 mL of blood. Hence, this consists of a seeding at 1/100^{th}.

### II.3. Counting Protocol.

In order to ensure the actual amount inoculated at t=0 s in the device, numbering on boxes is carried out. For this purpose, a volume of 0.1 mL of a dilution is sampled and then spread on a Petri dish with the rake.

This numbering is performed on the last two dilutions, in triplicate in order to be able to make an average.

### III. The Measurement Method.

The used electrochemical measurement method is potentiometry, i.e. a potential difference between a working electrode and a reference electrode, whose potential does not vary over time.

The device being composed of 8 working electrodes, the measurement is multiplexed: every 5 minutes, the potential of each electrode is recorded for 0.1 s.

This method allows obtaining 8 potential curves as a function of time. The experiment has been repeated with several different bacteria, several initial amounts and even without any bacterium in order to obtain negative controls providing information on the stability of the measurement.

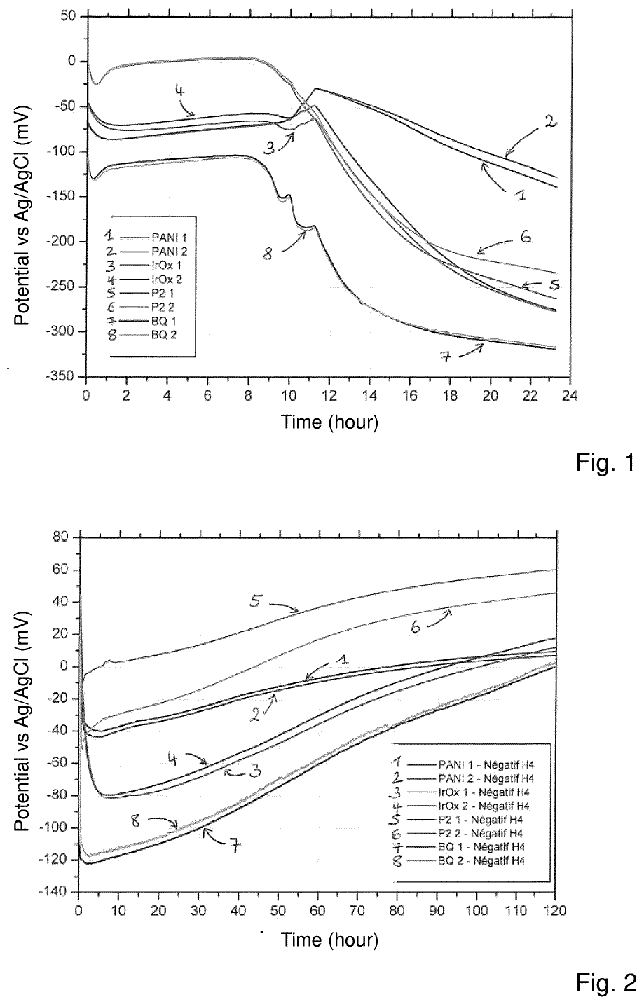

FIG. 1 shows the potentiometric curves obtained after having inoculated, at t=0, human blood with 38 cfu of Escherichia coli—ATCC 8739. 2 potential characteristic profiles are observed, a “polyaniline” profile where the potential increases and then decreases slowly, and a “carbon-based ink” profile where the profile drops more or less suddenly.

In view of the measured values, it is clear that the variation in the pH of the measurement medium it not solely detected but other electrochemical reactions, probably due to the secretion of redox metabolites, are also detected.

### IV. The Use of the Signals.

Since the biological media like the used blood is not inert, the electrodes record potential variations even without the presence of bacteria.

For a negative control of 5 days which corresponds to the standard time, in a hospital environment, to rule on the non-presence of bacteria, a slow drift of the potential is observed for all electrodes (cf. FIG. 2). These variations are very low compared to the experiments with bacteria.

In order to more clearly observe the start of the potential variations in the case of bacteriemias, the derivative of the smoothed potential at 5 points (sliding average over 5 points) for each electrode may be used. Thus, the rate of modification of the potential, much more relevant and readable than raw potentiometric curves, is observed.

Advantageously, the smoothed derivative of the potential is considered only after 4 h of recording. Indeed, the bacteria have an incompressible latency time during which it is not possible to consider any detection because the bacterial growth has not started yet.

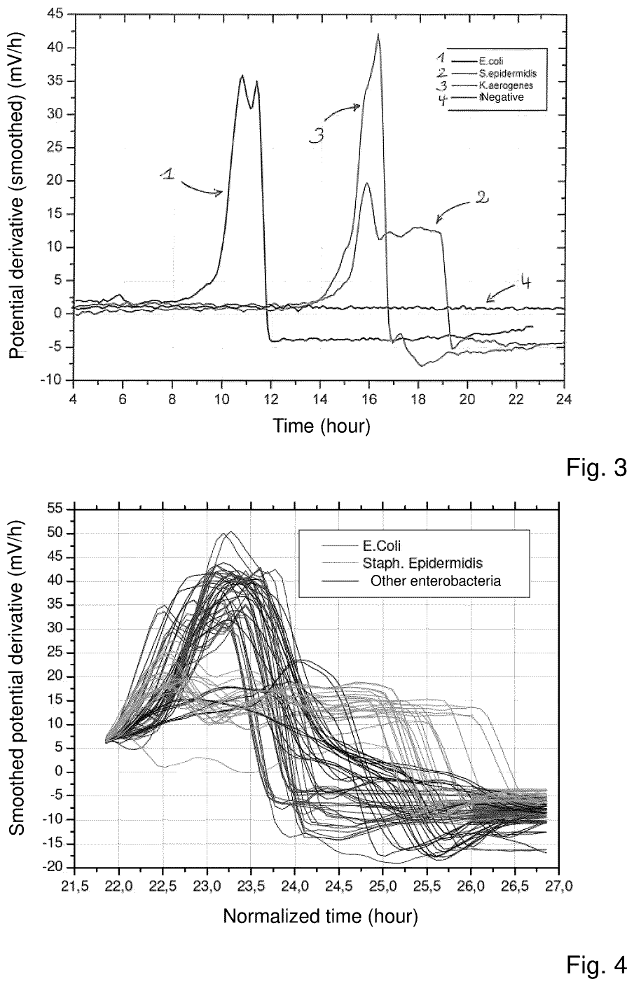

Hence, the curves shown in FIG. 3 are derivatives (smoothed over 5 points) of the potential, starting from t=4 h. As illustrated in this figure, the detection is clearly visible and it is possible to calculate a detection time.

In addition, with the polyaniline electrode, a characteristic profile of the curve is herein observed according to the detected bacterium. This difference in profile being repeatable, it is possible to consider using it to orient the identification of the bacteremia in parallel with detection thereof.

The differences in curve profile are visible on all electrodes but the polyaniline seems to be the most suitable one for identification, the differences being more obvious. On the other hand, the detection is clear for all electrodes, and even faster for electrodes based on carbon ink: iridium oxide, SunChemical Gwent C2070424P2 and Dupont BQ242.

### V. Creation of a Descriptor.

A descriptor from the different curves measured on polyaniline electrodes has been created. However, it is quite possible to consider creating a descriptor for each electrode.

This descriptor allows determining the bacterial species as a function of the profile of the curve of the smoothed derivative of the potential.

To do so, a database obtained from experiments carried out on human blood with different bacteria is used. The detail of these experiments is reported in Table 1 hereinafter.

TABLE 1

| | | Inoculum at t = 0 |
|---|---|---|
| Bacterium | Strain | in cfu |
| Escherichia coli | EC11 - ATCC 2739 | 10 |
| | | 10 |
| | | 100 |
| | | 100 |
| | EC21 - ATCC 35421 | 10 |
| | | 10 |
| | | 100 |
| | | 100 |
| | EC10 - ATCC 25922 | 10 |
| | | 10 |
| | | 100 |
| | | 100 |
| Staphylococcus epidermidis | SE26 - ATCC 12228 | 10 |
| | | 10 |
| | | 100 |
| | | 100 |
| | SE97 - ATCC 35984 | 10 |
| | | 10 |
| | | 100 |
| | | 100 |
| | SE101 - ATCC NCIMB | 10 |
| | 8853 | 10 |
| | | 100 |
| | | 100 |
| Klebsiella aerogenes | KA31 - ATCC 13048 | 10 |
| | | 10 |
| | | 100 |
| | | 100 |
| Enterobacter cloacae | EC8 - ATCC 13047 | 10 |
| | | 10 |
| | | 100 |
| | | 100 |
| Citrobacter freundii | CF7 - ATCC 8090 | 10 |
| | | 10 |
| | | 100 |
| | | 100 |

For each experiment, there are two polyaniline signals because there are two polyaniline electrodes per device. Hence, this represents a total of 36 smoothed derivatives of the potential as a function of time.

We start by suppressing the time dependence of the curves. By translation, the detection times are placed at the same time (cf. FIG. 4). Hence, for each curve, we obtain a given number of points (1 point every 5 minutes from 20 h to 25 h in FIG. 4).

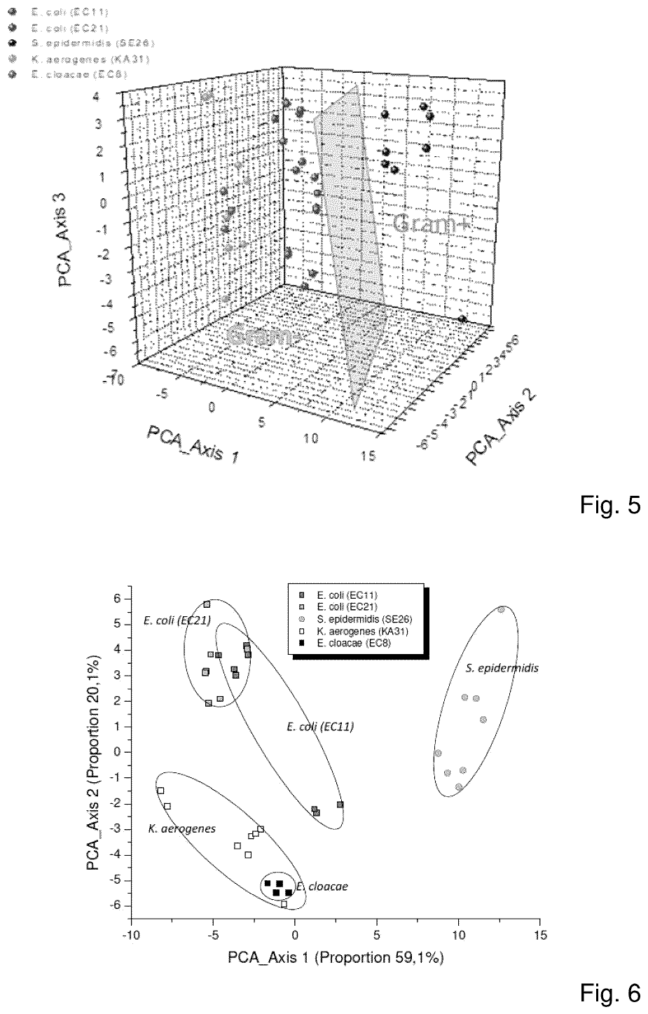

From these data, the Tanagra software may be used in order to perform an unsupervised principal component analysis (PCA). This analysis allows representing the different points in a 3D space (FIG. 5) or 2D space (FIG. 6).

In FIG. 5, quite distinct areas are observed for each bacterial group, confirming the relevance of this descriptor as to use thereof for orienting the identification of the detected pathogen.

With such a database to be completed with other bacteria, it is possible to discriminate Gram+ bacteria from the Gram− bacteria, and one could consider identifying the bacterial species.

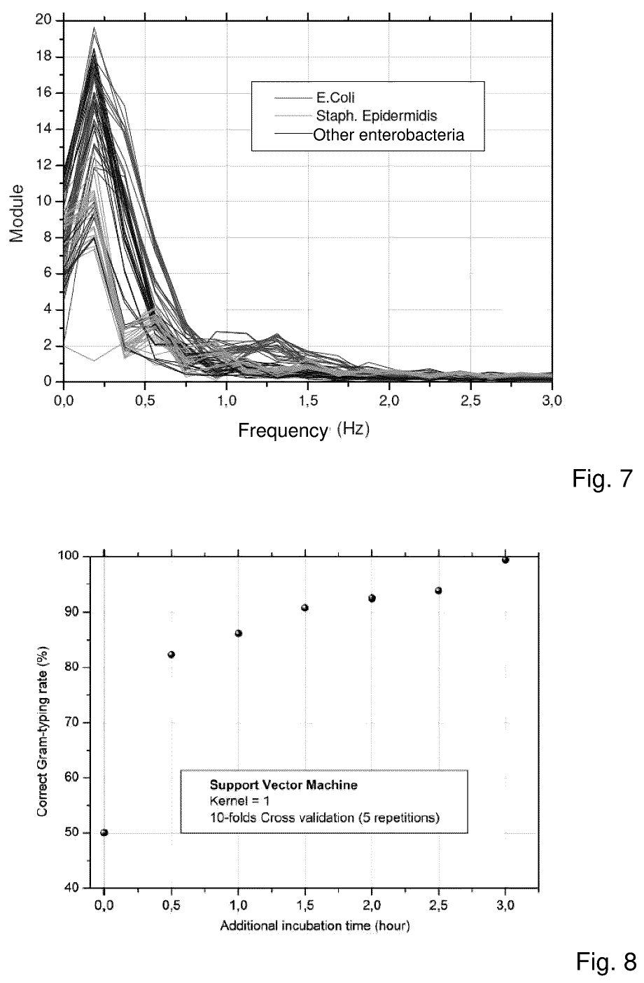

In order to reduce the number of dimensions of the vector, it is also possible to apply a Fourier transform to each curve shown in FIG. 4. Thus, for each experiment, 17 data corresponding to the 17 frequencies of interest between 0.1 and 2 Hertz are obtained as shown in FIG. 7.

A quite sharp difference is observed between the different groups of bacteria: E. coli/Other enterobacteriaceae (KA31, EC8 and CF7)/S.epidermidis.

Thanks to these data, it is possible to carry out supervised learning (or “support vector machine”) in order to obtain a confidence index as to the determination of the Gram of the bacterium as a function of time after the detection (FIG. 8).

Hence, the method according to the invention allows determining the Gram of the bacterium without any other manipulation and without any product, in contrast with a hospital reality where Gram staining is done under a microscope after addition of chemicals. Thirty minutes after detection, there is an accuracy of 82%, 2 h after detection, an accuracy of 90% and, 3 h after detection, an accuracy of 99%. The more information are obtained, the more it becomes possible to determine the nature of the detected pathogen.

It is possible to use only the potential difference without passing through the potential derivative and to replicate the same data processing.

### BIBLIOGRAPHICAL REFERENCES

[1] Shrestha et al, 2017, “The combined rapid detection and species-level identification of yeasts in simulated blood culture using a colorimetric sensor array”, PLoSONE 12(3): e0173130. doi:10.1371/journal.pone.0173130
[2] Templier et al, 2017, “Biochips for Direct Detection and Identification of Bacteria in Blood Culture-Like Conditions”, 7(1):9457. doi: 10.1038/s41598-017-10072-z.
[3] International application WO 2021/032654 A1 in the name of Commissariat a l'Energie Atomique et aux Energies Alternatives, published on February 25th, 2021.

1. (canceled)

2. A method for detecting and identifying a micro-organism contained in a sample, comprising
a) placing the sample, in a liquid culture medium, in the presence of at least one measurement electrode and a reference electrode and under conditions suitable for growth of the micro-organisms,
b) measuring, over time, a potential difference between said at least one measurement electrode and the reference electrode,
c) calculating a derivative of the potential difference from the measurement performed in step b) whereby a profile of the derivative of the potential is obtained, and
d) detecting and identifying a micro-organism contained in said sample from the profile obtained in step c).

3. The method according to claim 2, wherein said sample is selected from the group consisting of a biological fluid; water; a sample from a liquid industrial effluent; a sample originating from an air filtration; and a sample on an object.

4. The method according to claim 2, wherein said sample is blood.

5. The method according to claim 2, wherein said micro-organism is selected from the group consisting of prokaryotic micro-organisms, eukaryotic micro-organisms and other microscopic fungi and protists.

6. The method according to claim 2, wherein said at least one measurement electrode is selected from the group consisting of a solid electrode made of metal, a glass electrode, an electrode made of glassy carbon, an electrode made in the form of a deposit of a conductive ink and an electrode made in the form of an electrodeposit of a conductive element.

7. The method according to claim 2, wherein said reference electrode is made in the form of a deposit of an Ag/AgCl ink covered with a polymer layer.

8. The method according to claim 2, wherein said measurement of the potential difference during b) is done continuously.

9. The method according to claim 2, wherein
said measurement of the potential difference during b) is done discontinuously, and
b) consists in measuring the potential difference between said at least one measurement electrode and said reference electrode at a plurality of time points whereby potential measurement points are obtained.

10. The method according to claim 2, wherein said culture medium is subjected to stirring during a) and/or b).

11. The method according to claim 2, wherein said method comprises an additional step consisting in obtaining a normalized profile of the derivative of the potential from the profile of the derivative obtained in c).

12. A system for detecting and identifying a micro-organism contained in a sample likely to be implemented during the method as defined in claim 2 comprising
i) a device configured for cell culture in a liquid medium comprising at least one measurement electrode and a reference electrode,
ii) a unit for measuring a potential difference between said at least one measurement electrode and said reference electrode and configured to measure the potential difference, continuously and/or at a plurality of time points,
iii) an electronic unit for acquiring the measurement of the potential difference, connected to the potential measurement unit,
iv) an electric power supply unit for powering the electronic unit, and
v) a processing unit connected to the electronic unit and configured to process the measurement of the potential difference and to calculate, from the electronic unit, a derivative of the potential difference and including a module for detecting and identifying a micro-organism by analysis of the potential difference or by analysis of the derivative of the potential difference.
